(19) United States
(12) Patent Application Publication
(10) Pub. No.: US 2017/0248588 A1
(43) Pub. Date: Aug. 31, 2017

(54) POROUS MEDIUM WITH ENCODED REGIONS

(71) Applicant: Parallume, Inc., Los Gatos, CA (US)

(72) Inventor: Robert C. HAUSHALTER, Los Gatos, CA (US)

(21) Appl. No.: 15/503,684

(22) PCT Filed: Aug. 14, 2015
(86) PCT No.: PCT/US15/45425

§ 371 Date: Feb. 13, 2017

Related U.S. Application Data
(60) Provisional application No. 62/037,329, filed on Aug. 14, 2014.

(51) Int. Cl. G01N 33/543; G01N 33/545

(52) U.S. Cl. CPC G01N 33/54306; G01N 33/5436; G01N 33/545

(57) ABSTRACT

A method and system for identifying one or more analytes in a fluid includes preparing a porous medium having one or more pores and one or more encoded regions. The one or more encoded regions can contact internal pore surface or surfaces, external surface or surfaces, or any combination thereof, of the porous medium. The one or more encoded regions can include a capture probe or binding element which selectively captures the one or more of the analytes in the fluid. One or more of the one or more encoded regions can include at least one of physical properties and chemical properties which are different from the other one or more encoded regions.

### RELATED APPLICATIONS

This application claims claim the benefit of U.S. Provisional Application No. 62/037,329, filed Aug. 14, 2014, the entire disclosure of which is incorporated therein.

### FIELD

The present disclosure relates to encoding. In particular, the present disclosure is relates to encoding methods, devices and compositions that are used for determining the presence and amount of one or more analytes of interest in a fluid within a porous solid.

### BACKGROUND

There are a very large number of methods, which may be employed to determine the nature and amount of an analyte material present in a solution. Many diagnostics assays determine the amount of an analyte, which is an indicator of some particular state or condition in the patients. One common method to characterize the analytes is to use a lateral flow assay where the presence of the analyte is determined by the localization (onto a specific location) and detection of a nanoparticle, which has been derivatized with a capture probe which binds the analyte of interest. Difficulties in synthesizing and manipulating large numbers of different nanoparticles have heretofore precluded the development of multiplexed assays of this type where many analytes are determined simultaneously instead of one or a few analytes at a time. There is, therefore, an urgent need to increase the number of assays that can be performed at the same time (i.e., increase the multiplexing level of the assay), which would increase the number of assays performed for a given cost and time, by a method that does not employ any nanoparticles of other solid state materials but uses only soluble molecular reagents.

Therefore, a method and system for determining the presence and amount of one or more analytes of interest in a fluid is needed, which addresses the above-mentioned problems of existing methods and systems and that is capable of simultaneously sensing, detecting and characterizing an arbitrarily large number of different analytes in a multiplexed fashion.

### SUMMARY

Disclosed herein is a method for sensing, identifying, and characterizing one or more analytes in a fluid. In various embodiments, the method comprises preparing a porous medium comprising one or more pores, the porous medium including one or more encoded regions. The one or more encoded regions can contact internal pore surface or surfaces, external surface or surfaces, or any combination thereof, of the porous medium. The one or more encoded regions can comprise a capture probe or binding element which selectively captures the one or more of the analytes in the fluid. One or more of the one or more encoded regions can comprise at least one of physical properties and chemical properties which are different from the other one or more encoded regions. The method further comprises filling the pores of the porous medium with the fluid containing the one or more analytes. The one or more analytes can contact the one or more encoded regions of the porous medium, wherein the capture probe or binding element of each of the encoded regions selectively captures the one or more analytes. The method further comprises characterizing the one or more analytes captured by each of the one or more encoded regions.

Further disclosed herein is a system for sensing, identifying, and characterizing one or more analytes in a fluid. The system, in various embodiments, comprises: a porous medium comprising one or more pores; one or more encoded regions, wherein the one or more encoded regions contact the internal pore surface or surfaces, external surface or surfaces, or any combination thereof, of the porous medium, and wherein the one or more encoded regions comprises at least one of physical properties and chemical properties which are different from the other encoded regions; and a capture probe or binding element disposed on or in the one or more encoded regions, the capture probe or binding element for selectively capturing the one or more of the analytes in the fluid. In operation, the pores of the porous medium are filled with the fluid containing the one or more analytes, the one or more analytes contacting the encoded regions of the porous medium, wherein the capture probe or binding element of each encoded region selectively captures the one or more analytes; and wherein the one or more analytes captured by each encoded region is characterized.

In some embodiments, the porous medium can be made from a material comprising an organic polymer, a glass, a mineral, a paper, a wood, an oxide, a carbohydrate, agarose, a metal or any combination thereof.

In some embodiments, encoding of the one or more encoded regions can be based on a physical property, a chemical property, a magnetic property, or any combination thereof.

In some embodiments, the physical property can comprise a shape, a size, a color, emission or absorption behavior, the number of voids, the number of indentations, the number of channels, the number of markings or protrusions, the scattering of electromagnetic radiation, or any combination thereof.

In some embodiments, each of the one or more analytes can comprise an organic molecule, a biochemical molecule, a nucleic acid, a peptide, a protein, an antibody, a drug candidate molecule, an anionic and cationic inorganic material, a radioactive species, or any combination thereof.

In some embodiments, an encoding material can be used for generating binary, absolute and ratiometric optical codes within the encoded regions, or any combination thereof.

In some embodiments, the encoding material can comprise a Raman-active material, an organic dye, an inorganic pigment, a lanthanide material, a rare earth material, quantum dots, a vapor deposited thin film material, photonic crystals, or any combination of thereof in any ratio.

In some embodiments, the encoded regions can be encoded with self-luminescent materials including laser diodes, chemiluminescent light emitting diodes, lasers, filaments or materials that transpond like RFID tags, or any combination thereof.

In some embodiments, the characterizing of the one or more analytes includes determining the amount of the one or more analytes captured by each of the one or more encoded regions, the determining of the amount can comprise contacting a reporter material with the one or more analytes captured with the capture probe or binding element of each of the one or more encoded regions and measuring the level of the reporter material on each of the one or more encoded regions to determine the amount of each of the one or more analytes bound to the capture probe or binding element of that encoded region.

In some embodiments, the contacting of the reporter material with the one or more analytes comprises labeling, attaching or derivitizing the one or more analytes with the reporter material.

In some embodiments, the one or more encoded regions are encoded with one or more lanthanide materials.

In some embodiments, the system further comprises a reporter material for determining the amount of each of the one or more analytes bound to the capture probe or binding element of that encoded region.

### BRIEF DESCRIPTION OF THE DRAWINGS

FIGS. 1A-1D illustrate an embodiment of a system or assay, comprising a porous medium having one or more differently encoded regions, for determining the presence, nature, and amount of one or more different analytes of interest in a fluid according to the present disclosure If the one or more analytes are not directly detectable, then the one or more analytes are treated with a reporter material after being selectively captured on the one or more encoded regions which renders the analyte detectable.

FIGS. 2A-2B illustrate another embodiment of the system, where the one or more analytes are treated with a reporter material prior to being selectively captured on the one or more encoded regions.

FIGS. 3A-3F illustrate another embodiment of the system, where the porous medium comprises a sheet or layer of porous material and includes a plurality of differently encoded regions contacting an exterior surface of the sheet or layer of porous material and where fluid laterally flows through the porous medium in a direction which is parallel to the exterior surface.

FIGS. 4A-4G illustrate another embodiment of the system, which is similar to the embodiment of FIGS. 3A-3F, but where the plurality of differently encoded regions are disposed or embedded within the porous region.

FIGS. 5A-5G illustrate another embodiment of the system, which is similar to the embodiment of FIGS. 3A-3F, but where the fluid flows through the sheet or layer of porous material from a first exterior surface thereof, which is contacted by the plurality of differently encoded regions, to a second exterior surface thereof that is opposite to the first exterior surface.

FIGS. 6A-6F illustrate another embodiment of the system, wherein fluid radially flows through the porous medium in an outwardly radial direction.

### DETAILED DESCRIPTION

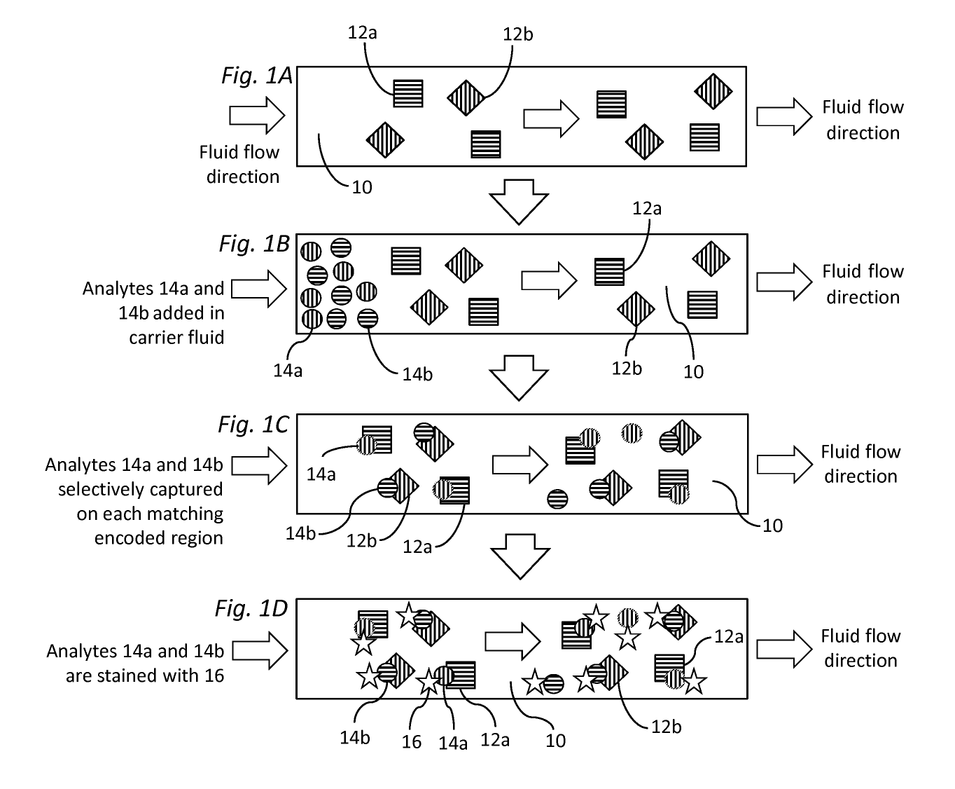

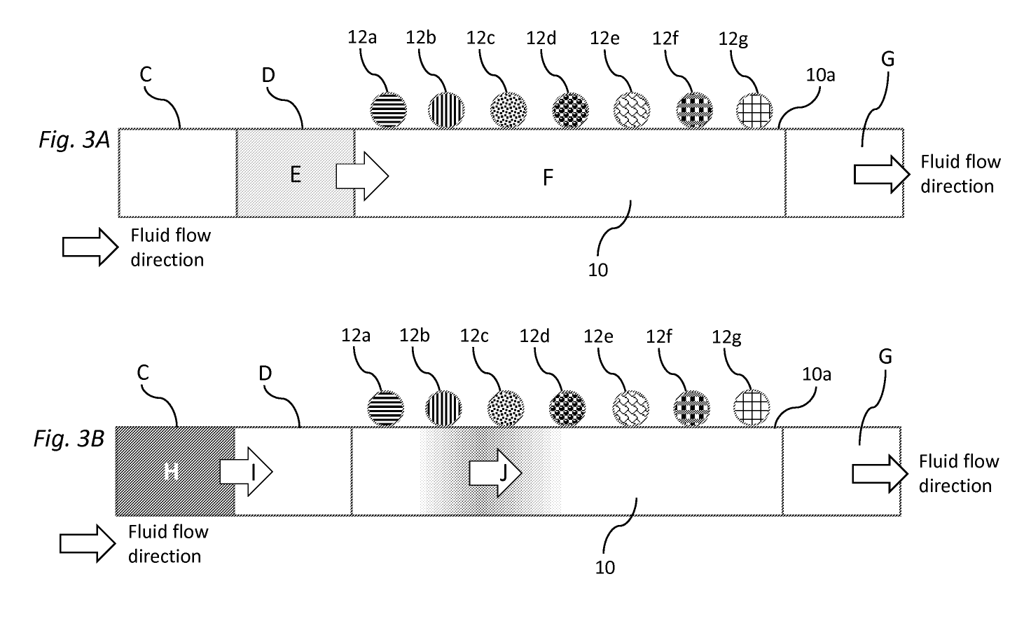

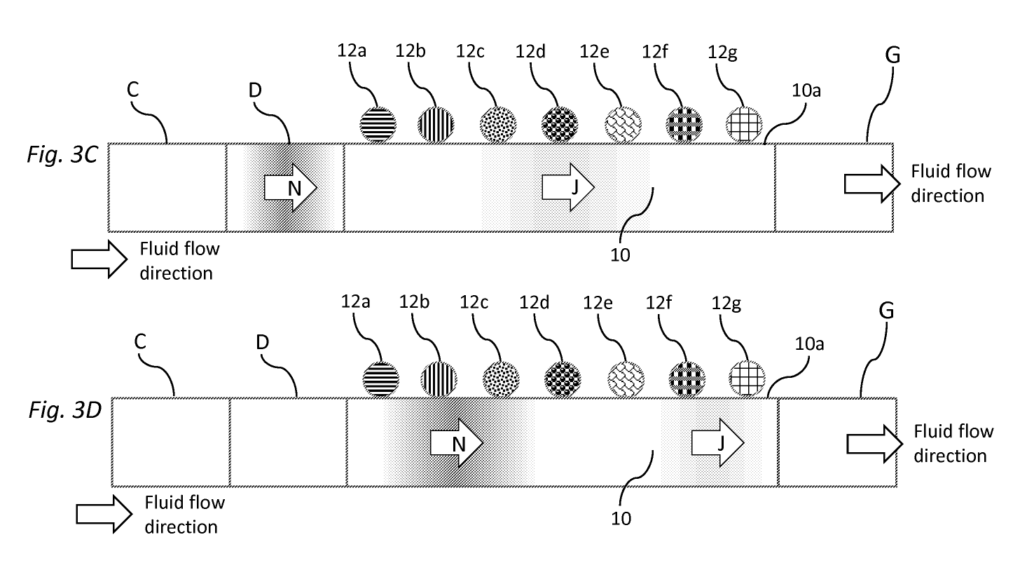

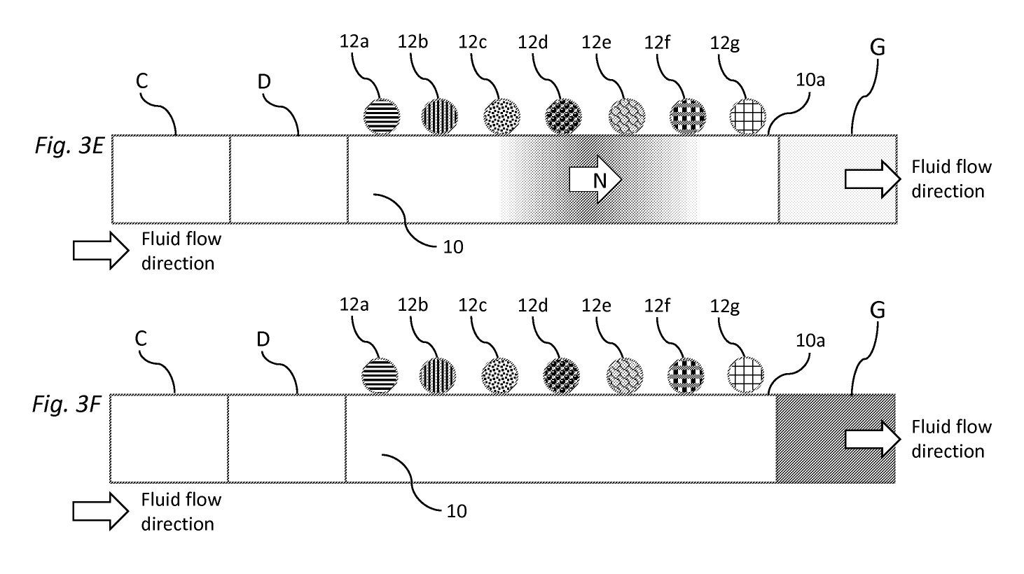

The present disclosure presents systems, methods, and compositions for determining the presence and amount of one or more different analytes of interest in a fluid. The system and method comprise a porous solid or medium 10 through which the fluid flows, an embodiment of which is illustrated in FIGS. 1A-1D. The porous medium is provided with one or more differently encoded regions 12a, 12b. Each encoded region is endowed with a unique, encoded chemical or physical property. The unique encoding allows the region to be either (a) distinguished from other encoded regions which have been encoded in a different fashion or (b) grouped with regions which have been encoded in the same fashion. In the context of the present disclosure, each of the encoded regions 12a, 12b is a particular region, particle, object or other distinguishable entity, provided with the porous medium 10 such that encoded region 12a may be distinguished from encoded region 12b because encoded regions 12a and 12b are encoded differently from one another, with first and second different codes. The one or more encoded regions 12a all have the same first code and the one or more encoded regions 12b likewise all have the same second code and the codes for encoded regions 12a are distinguishable from those of encoded regions 12b. The one or more encoded or otherwise uniquely identifiable regions 12a,12b can be disposed or embedded in the porous medium 10 (FIGS. 1A-1D), or on or in contact with the porous medium 10 (FIGS. 3A-3F). In other embodiments, one or more of the encoded regions 12a, 12b can be embedded in the porous medium 10 and one or more of the other encoded regions 12a, 12b can be provided on or in contact with the same porous medium 10. As illustrated FIG. 1B, the porous medium 10 can contain a fluid within or on the pores or surface thereof in which one or more different analytes or materials of interest 14a, 14b are dissolved or suspended, are used to detect, measure and identify one or more different analytes or materials of interest 14a, 14b.

Referring to FIG. 1A, each of the encoded regions 12a, 12b is derivatized or associated with a distinctive capture probe or binding element that uniquely detects the presence, absence or amount of one or more analytes and largely excludes and does not detect other analytes present. Other different encoded regions within the same porous medium detect a different analyte or analytes. Each of the encoded regions 12a, 12b can be differentiated from other encoded regions 12a, 12b by a unique physical or chemical property (including but not limited to a shape, a unique optical emission code emitted when excited, a unique size, etc.), or a combination of more than one type of encoding, that distinguishes it from all other encoded regions 12a, 12b. A known location of a given region within the porous medium 10 is not a physical or chemical property of that region so such a region is not encoded within the context of the present disclosure. The encoded regions 12a, 12b may occupy random positions such that their locations within the porous medium 10 relative to the medium 10 and one another are not necessarily known before the measurement of the analytes. Each of the encoded regions 12a, 12b possesses a unique capture probe, binding region or other structure that selectively captures one of more analytes from the fluid within the porous medium 10. Therefore, by (a) differentiating the encoded regions 12a, 12b within the porous medium 10 from one another, (b) knowing which of the encoded regions 12a, 12b identifies which particular analyte and (c) having the ability to determine to what extent which of the encoded regions 12a, 12b has changed in response to the capture or detection of that encoded region's unique analytes, all analytes in the fluid may be simultaneously identified, or identified in rapid succession, as the fluid moves through the porous medium 10, and the amount of all analytes present is thereby determined.

There are many advantages of using the combination of the porous medium with the one or more encoded regions for the identification of analytes. One important function of the porous medium is to provide immobile scaffolding through which the analyte-containing fluid moves thereby allowing intimate contact, diffusion, convection, mixing and conduction of the fluid into, onto, through and out of the encoded regions within the porous medium. Using the porous medium to hold the reagent and solvents within the fluid eliminates the need for complex fluid handling (i.e., pipets, stirrers) as both the addition and movement of the fluid may be accomplished passively by evaporation, capillary action or gravity. Unlike particles in a liquid, there is no need to perform a separation of the particles from solution in order to measure of image them as the encoded regions are part of the solid porous medium and may be measured directly without further manipulation. Another useful attribute of the encoded regions within a porous medium is that the encoded regions are physically separated from one another on or within the porous medium which allows each encoded region to be measured separately without interference from other encoded regions. Movement through the pores mixes the fluid so a uniform sample is presented to the encoded regions for analysis and results in lower variance when comparing an encoded region to another region of the same optical code. The porous medium provides a high surface area which allows a large number of encoded regions to be available per unit volume of fluid. The fluid within the pores cannot be spilled and less susceptible to evaporation. The porous medium holds the encoded regions in a fixed position thereby facilitating the analysis of the analytes by image acquisition.

The fluid can be any gas, liquid, suspension, ionic liquid, liquid metal, colloid, molten salt, liquid solution or combination of gases and liquids, in which the analytes or materials of interest are dissolved or suspended. As illustrated in FIGS. 1B and 1C, when the moving fluid containing the analytes 14a, 14b contacts the capture probe-derivatized encoded regions 12a, 12b within or on the porous medium 10, each of the fluid-contacting encoded regions 12a, 12b simultaneously react with their specific targets analytes 14a, 14b when the analyte is present. To detect, identify, measure or quantitate the analyte 14a, 14b within the fluid, a given one of the analytes 14a and 14b must bind to, change or otherwise interact with the corresponding one of the encoded regions 12a and 12b, which is acting as a specific receptor for this particular analyte. The bound analyte 14a, 14b (FIG. 1C) may then be detected by the change it directly induces within the encoded region 12a, 12b (e.g., changes the color of the encoding region upon binding). The encoded region 12a, 12b itself may detect the analyte or, alternatively, another species, (e.g., an antibody that could capture the analyte of interest) can be placed in, on or in proximity to the encoded region which interacts with the analyte 14a, 14b. On the other hand, the bound analyte 14a, 14b may be treated with a “staining” material 16 (i.e., a dye or reporter material), as illustrated in FIG. 1D, which produces a change in the physical properties of the encoded region such as a change in the size, color, optical density, reflectivity, emissivity, orientation, shape, magnetic moment, thermal or electrical conductivity, etc. or a release or consumption of detectable chemical species.

In addition to containing the analyte, other fluids, such as those containing reporter materials, washing materials, secondary antibodies or materials used to enhance or develop the reporter or encoded region may be used.

The fluid can be caused to move within the pores of the porous medium by any means including, for example, gravity, pressure differential, electroosmosis, displacement with another fluid, heat, capillary action, wicking, chemical reactions, physical force, centrifugation, spinning, a temperature differential or evaporation.

The porous medium can be a solid which contains one or more of pores, channels, holes and other void spaces (hereinafter “pores”) where none of the solid or liquid material comprising the scaffolding (i.e., non-void space) of the porous solid exists. In other embodiments, the porous medium can be a tube (single pore). The pores may range in size from picometers to millimeters in diameter, length or other dimensions, may have regular or irregularly-shaped pores and may possess a narrow or wide distribution of pore sizes. The porous medium can be any type of material where there are pores of a size smaller than the porous object itself. The pores may or may not communicate with the porous medium's external environment and the pores may or may not interconnect with one another. Materials from which porous media are composed include either a monolithic solid with internal pores, a porous material resulting from the packing together of particles, pieces or spheres of a solid, non-porous material or a combination of these two types.

The porous medium can be a synthetic material such as an organic polymer, foam, gel, a glass, a metal, a ceramic, an inorganic material or an amorphous material or crystalline solid. The porous medium can be other materials such as agarose, carrageenan, gums, minerals such as zeolites, diatoms, wood, cellulose, nanocellulose, nanocrystalline cellulose, nitrocellulose, plant materials and porous rocks. The pores in the solid can be formed by any method including chemical, physical or other means during the synthesis or casting of the porous medium, during the preparation of the synthetic precursors to the porous medium or after the synthesis or fabrication of the porous medium is complete. The pores can all be of the same size within a single porous medium, a narrow range of sizes or an indefinite, wide or random distribution of pore sizes.

The morphology, form or structure of the porous medium through which the fluid containing the analytes moves can be of any two-dimensional or three-dimensional shape or form including films of any thickness, ribbons, sheets, tubes and solids with any cross sectional shape including round, rectangular, square, trapezoidal, a random shape or combinations of these shapes or other shapes.

The encoded regions within the porous medium may be encoded and differentiated from one another by their shape, size, absorption, excitation or emission spectra, porosity, reflectivity, inductance, electrochromism, piezo- or pyroelectric response, electrical conductivity, scattering, Raman scattering, patterns of occluded particles or voids, magnetic properties, number of holes, indentations, channels or other physical features, a change induced by any type of external stimulus such as temperature changes, electric fields or irradiation with electromagnetic radiation or a change induced by binding of a particular analyte. The encoded regions may be differentiated from one another by any suitable means either before or after interaction of the analytes or analytes and reporters within the fluid in the pores or on the surface of the porous medium. Since each encoded region possesses a specific capture probe or binding site that preferentially binds one more specific analytes, once the identity of an encoded region is known, the identity of the analyte that binds to that particular encoded region is likewise known. One preferred method for determining the amount of the analyte bound to the encoded region is to stain the analyte with a reporter dye and subsequently measuring the amount of dye present in the encoded region or the amount compared to some standard or control analyte within or external to the porous medium.

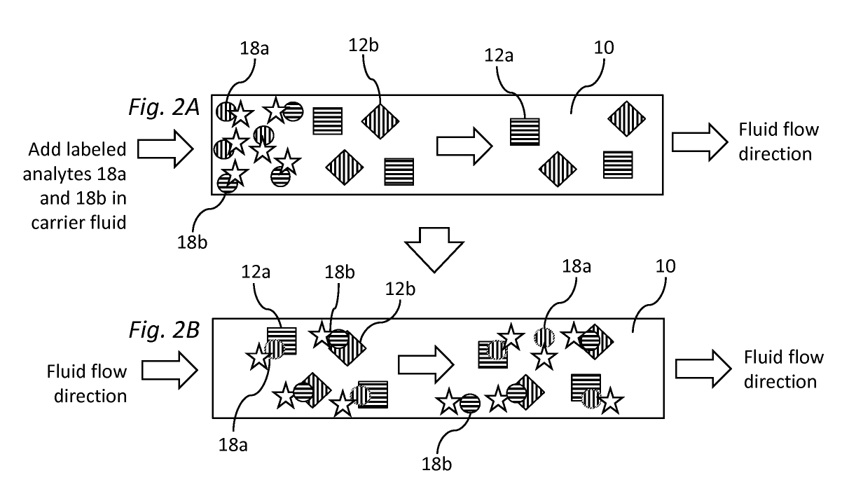

The encoded regions may be identified optically by any type of absorption, emission or scattering of electromagnetic radiation. One preferred method to encode the encoded region is to employ optical encoding where an emission of electromagnetic radiation that comprises a unique, exclusive and distinct optical code, optical barcode or spectral signature is induced by suitable excitation of the emitting species or emitters. For example, a simple type of optical encoding is illustrated in FIGS. 1A-1D, wherein the two encoded regions 12a and 12b within the porous medium 10 are distinguished from one another in FIGS. 1A-1D based on their striping pattern (i.e., either horizontal or vertical stripes), respectively. Therefore, one can distinguish the two different and separate encoded regions 12a, 12b by their different emitted, transmitted or reflected optical pattern. Once the encoded region 12a, 12b has been identified by its encoding (in this case the striping pattern), the analyte 14a, 14b that binds to that particular region 12a, 12b, is also known, since the identity of the capture probe that binds a given analyte 14a, 14b associated with a particular encoded region 12a, 12b is known prior to the beginning of the analysis. Unless the analytes 18a, 18b were stained prior to the analysis as illustrated in FIGS. 2A and 2B, staining of the analytes 14a, 14b with a reporter stain 16, and measuring the strength of the reporter signal obtained for that particular encoded region 12a, 12b, provides information on the amount of the analyte 14a, 14b present in the fluid within the porous medium, as illustrated in FIG. 1D.

Each encoded region selectively detects one or more analytes of interest and largely excludes other analytes. The selectivity of the encoded region toward a particular analyte may be an inherent part of the encoded region itself or the encoded region may be derivatized, combined or otherwise associated with another entity that functions as a selectively capture probe or binding site for the analyte. As an example of inherent selectivity, the encoded regions may be composed of different zeolites embedded in a porous silica matric (i.e., the porous medium), where each encoded region may be distinguished from other zeolite-encoded regions by their different x-ray diffraction patterns, each of which may selectively absorb one of several different small gaseous molecules in a mixture preferentially over other molecules in the mixture. When the porous medium with encoded regions is exposed to the mixture of gaseous molecules, each small molecule is selectively absorbed preferentially into one of the different zeolites within the zeolite-containing encoded regions. When the molecule is absorbed, the x-ray diffraction pattern of the zeolite changes in proportion to the amount of the molecule absorbed thereby allowing the determination of the amount of that particular molecule in the gaseous mixture. As an example of a derivatized or chemically functionalized encoded regions, three different color encoded regions within a porous solid are derivatized by attaching a different antibody onto each different color-encoded region. These three antibodies selectively bind three different antigens out of a serum sample. When the fluid containing the analytes (i.e., the three antigens) passes through the porous medium and contacts the antibody-derivatized encoded regions each antigen binds to its complementary antibody. If required, subsequent staining with a reporter 16 identifies the amount of each analyte 14a, 14b, 18a, 18b on each encoded region 12a, 12b, as illustrated in FIGS. 1D and 2A.

Referring to FIGS. 1A-1D, the encoded regions 12a, 12b can be used to simultaneously identify multiple analytes 14a, 14b in the fluid within the porous medium 10. When one encoded region (e.g., encoded region 12a) reacts with and identifies one or more target analytes (e.g., analyte 14a), another, different and separate encoded region (e.g., encoded region 12b) simultaneously identifies one or more different target analytes (e.g., analyte 14b). The embodiment illustrated in FIGS. 1A-1D, depicts two encoded regions 12a and 12b that have been encoded using striping patterns (which represent encoding)—in this case encoded region 12a may be encoded with horizontal stripes and encoded region 12b is encoded with vertical stripes. The two encoded regions 12a, 12b are distinguished in this case by their different optical appearance (i.e., vertical stripes vs. horizontal stripes). In FIG. 1B, the fluid, which contains analytes 14a and 14, moves through the porous medium 10 and encoded region 12a selectively binds analyte 14a and encoded region 12b selectively and simultaneously binds analyte 14b, as shown in FIG. 1C. To analyze the results of this example, the encoded regions 12a and 12b are first identified by their encoding (either horizontal or vertical stripes. Then, the encoded regions 12a and 12b have either been: (a) derivatized with a capture species or capture probe that selectively binds the analyte 14a, 14b of interest to the exclusion of other species; or (b) the encoding region itself can selectively bind the analyte 14a, 14b of interest. The amount of bound analyte 14a, 14b, or a detectable change induced in the encoding region 12a, 12b, is determined, for example, by measuring the amount of a reporter dye molecule 16 on the analyte 14a, 14b, or by the amount of a reporter dye molecule 16 that can be placed onto the analyte 14a, 14b once bound to the encoding region, as illustrated FIG. 1D. Therefore, determining the amount of the reporter 16 on a given encoding region 12a, 12b gives the amount of the particular analyte 14a, 14b (via interrogation of the reporter signal) captured on that specific encoding region. Although the embodiment of FIGS. 1A-1D, illustrates two encoded regions 12a and 12b, other embodiments of may have any number of encoded regions and any number of analytes, where the number of analytes and/or encoded regions is limited only by the number of distinguishable encoded regions and the specificity and sensitivity of the encoded regions for capturing their particular analyte from among all analytes in the fluid.

In some cases, the encoded region may specifically bind the one or more analytes present in the fluid within the porous medium with the exclusion of other analytes and the analyte may change the physical or chemical properties of the encoded regions upon interaction—or the analyte and encoded region could interact via electromagnetic radiation, magnetic interactions or optical energy transfer (e.g., FRET). The encoded region may be an integral part of the porous medium, within the interior of the porous medium or attached to the surface or pores of the porous medium. The encoded region may be incorporated into the porous medium during fabrication of the porous medium or added into the pores or surface of the porous medium after fabrication of the porous medium.

The reporter (e.g., a dye molecule), which, by binding to the analyte, reveals how much of the analyte has bound to or changed the encoded region (e.g., by the intensity of its color) of the analytes, can be added (reporter 16) either after the analyte 14a, 14b has bound to the encoded region 12a, 12b (FIG. 1D) or attached to the analyte 18a, 18b before the analyte-containing fluid contact the porous medium 10 with encoded region (FIG. 2A). The reporter can be any material that can be distinguished from the porous medium and the fluid within the pores of the porous medium. Examples include organic dyes, phosphors, rare earth emitters, quantum dots, Raman active materials and nanoparticles.

Each encoded region binds a specific analyte or group of analytes and largely excludes other materials or analytes within the fluid. In the embodiment of FIGS. 1A-1D, the analytes 14a, 14b are only bound by their own specific encoded region, i.e., analyte 14a binds to encoded region 12a but not to encoded region 12b. In addition to the encoding itself that is present within each encoded region, the encoding region may contain another species in close association with the encoded region that provides the specificity for the desired analyte. For example, an antibody against an analyte of interest could be associated with a uniquely encoded region. Only the analyte of interest would bind to that particular encoded region.

Referring now to FIGS. 3A-3F, the different encoded regions 12a-12g can be present, contact or lie upon on an exterior surface 10a of the porous medium 10 through which the analyte-containing fluid passes. In FIGS. 3A-3F, areas C and D of the porous medium 10 are fluid loading areas where, for example, sample E is added to porous medium area D and reporter stain (H), which will bind to the analyte that has been captured on the encoded region upon encountering it, is added to porous medium area C. As the fluid flows (FIG. 3B) in the direction shown as indicated by the right-pointing arrows I and J, the analyte in sample E of the flowing fluid passes through area F of the porous medium 10 and makes contact with encoded regions 12a-12f. The sample E within the fluid flows through the porous medium area F by diffusion, convection, turbulent mixing and other means. The fluid moves through area F of the porous medium 10 to absorbent area G of the porous medium 10, thereby removing the fluid from the porous medium 10 to initiate and maintain fluid flow therethrough with area G acting as an absorbent sink for the fluid moving through the porous medium 10.

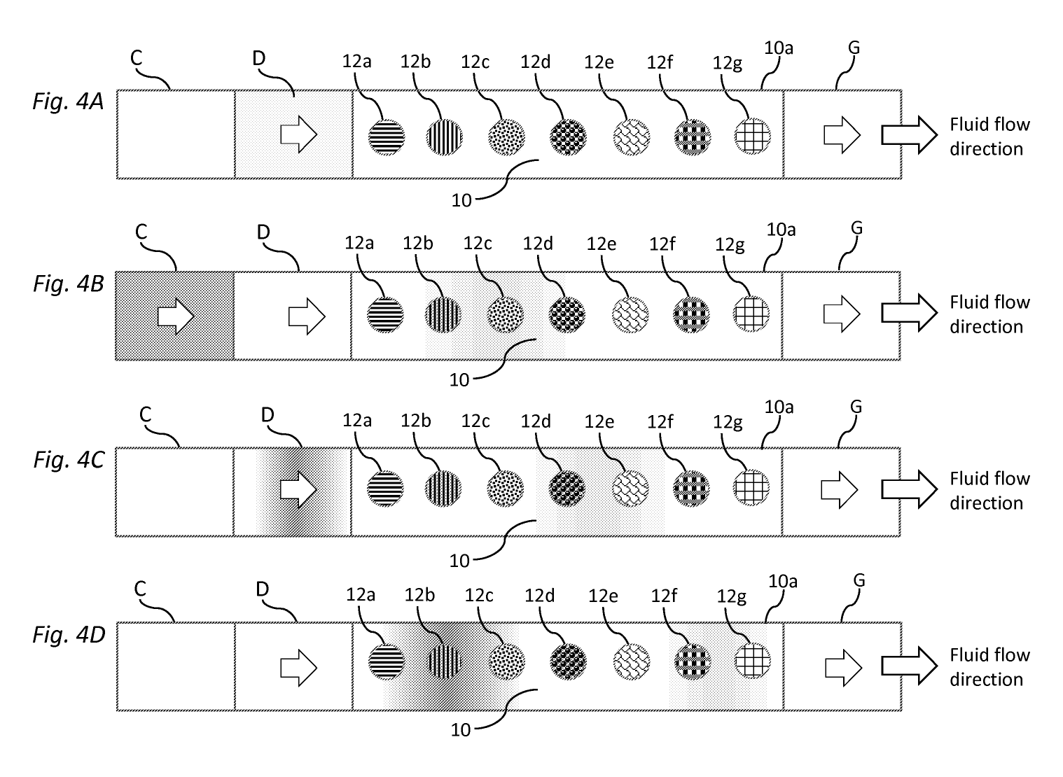

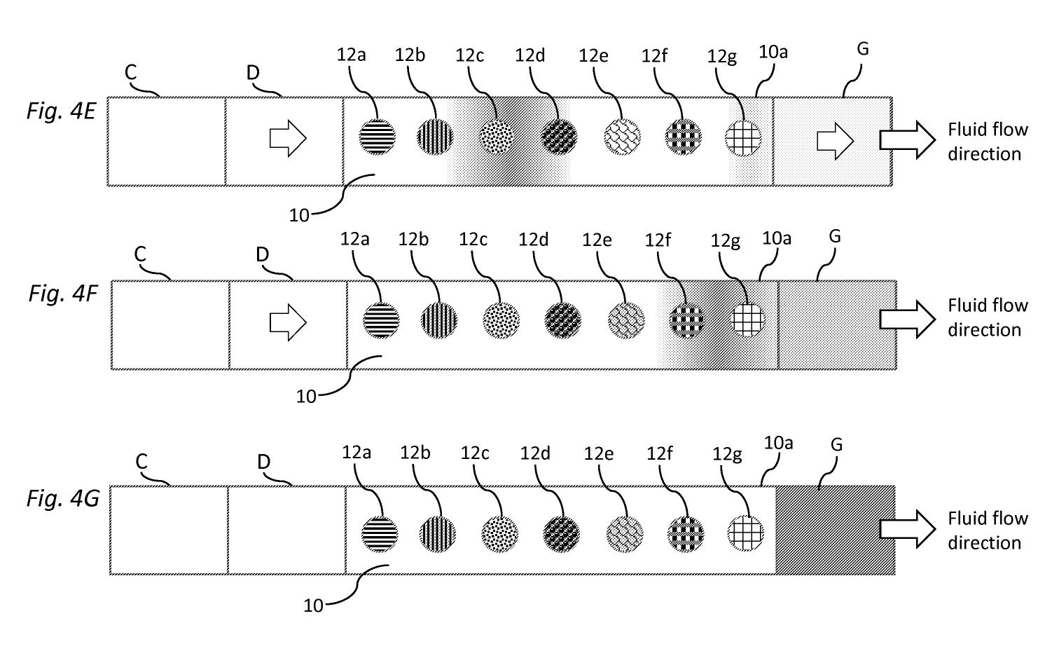

Rather than the encoded regions 12a-12g contacting an external surface of the porous medium 10, the encoded regions 12a-12g may contact the porous medium 10 by contacting an internal surface or by embedding of the porous regions 12a-12g into the porous medium 10 as shown in FIGS. 4A-4G where the elements shown therein are identified with the same reference characters used in the embodiment of FIGS. 3A-3F to identify similar and/or like elements shown therein.

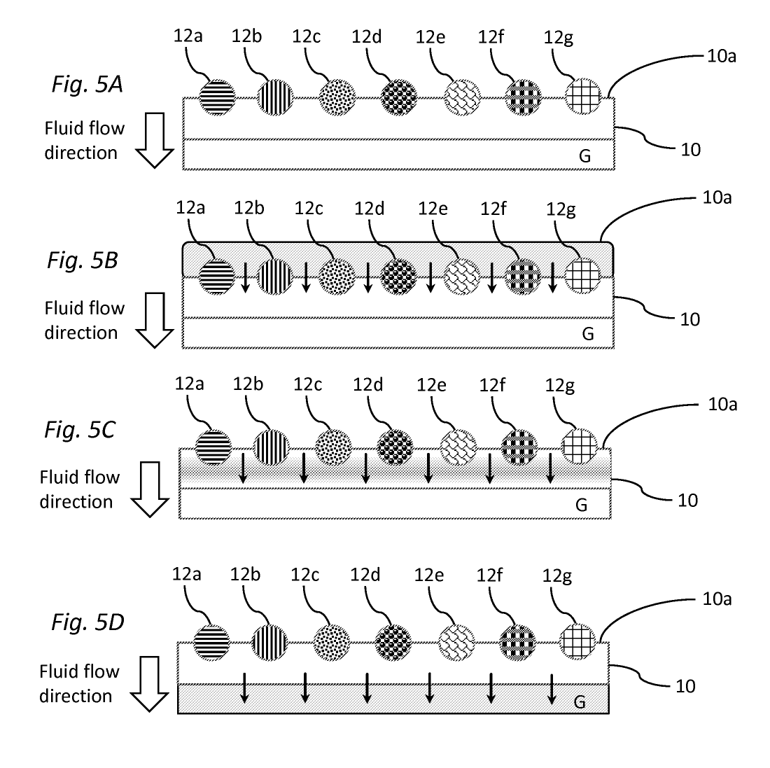

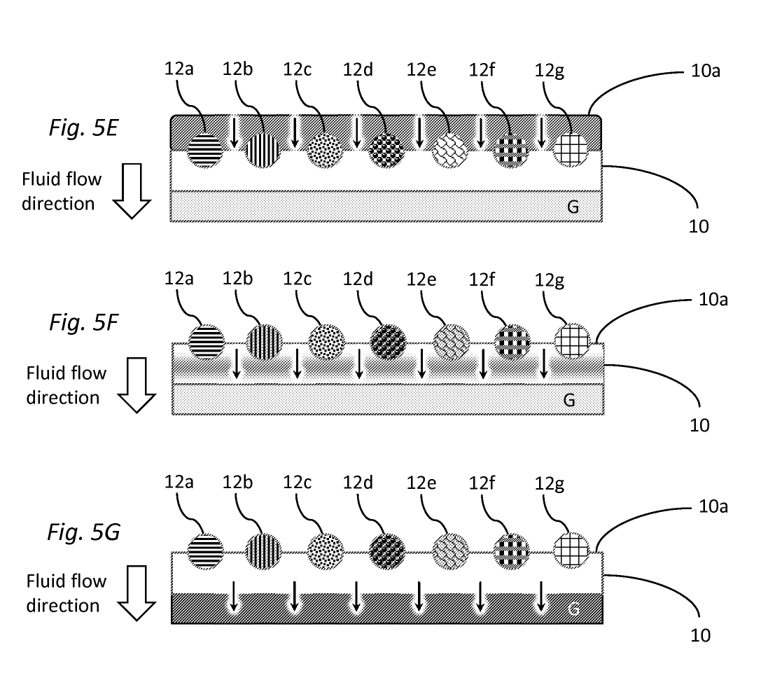

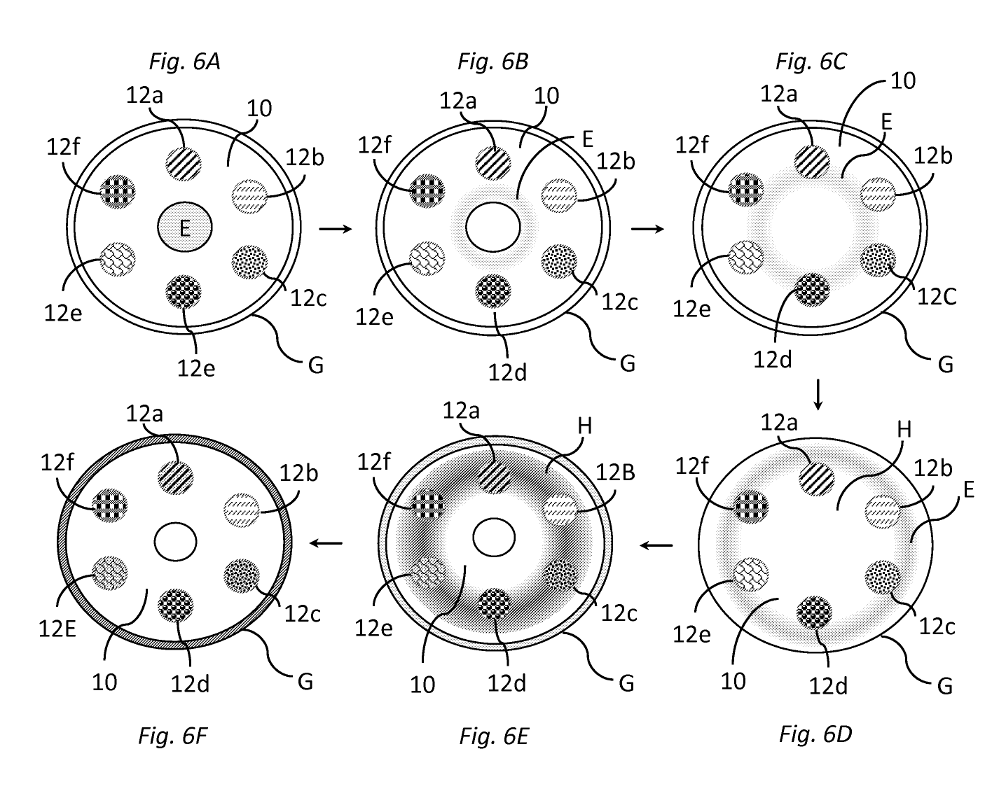

As shown in FIGS. 3A-F and 4A-G, the analyte-containing fluid can flow any direction relative to the length and width of the porous medium 10 but not along the thickness dimension of the layer, sheet, coat, stratum or film-like structure of the porous medium 10. The analyte-containing fluid may also pass through the layer, sheet, coat, stratum or film-like structure of the porous medium 10 in a direction perpendicular to the plane of the layer, sheet, coat, stratum or film-like structure of the porous medium 10 as illustrated in the embodiment of FIGS. 5A-5G, where the elements shown therein are identified with the same reference characters used in the embodiment of FIGS. 3A-3F to identify similar and/or like elements shown therein. The fluid may also move through the porous medium 10 in a radial direction relative to the porous medium 10, as illustrated in the embodiment of FIGS. 6A-6F, where the elements shown therein are identified with the same reference characters used in the embodiment of FIGS. 3A-3F to identify similar and/or like elements shown therein.

Referring again to FIGS. 3A-3F, and in particular, to FIG. 3C, the encoded regions 12c and 12d that have contacted the analyte-containing sample E, but have not yet contacted the reporter stain H (arrow N) are present upon the surface 10a of the porous medium 10. Ultimately, some of the encoded regions 12a-12g bind their targeted specific analyte from the fluid if the analyte is present in the sample. More specifically, as the fluid (arrow N), which contains the reporter stain H specific to the analyte bound to the encoded regions, moves through the porous medium 10, the reporter stain H penetrates encoded region 12a (FIG. 3C) and encoded region 12b (FIG. 3D) by diffusion, convention and turbulent mixing. As illustrated in FIGS. 3E and 3F, the encoded regions which have captured their specific target analyte will retain the reporter stain H (encoded regions 12b, 12d,12e) while encoded regions 12a, 12c, 12f, and 12g, for which there was no analyte present in the fluid do not significantly retain any reporter stain. This lack of reporter signal in these encoded regions means that the analyte associated with that particular region is not present in the fluid.

As illustrated in FIG. 3F, after both the sample E-containing fluid and the reporter stain H have moved through area F of the porous medium 10 and into the absorbent area G of the porous medium 10, encoded regions 12b, 12d, and 12e that have retained an analyte that can be stained by the reporter H are apparent and the amount of analyte retained within encoded regions 12b, 12d, and 12e is proportional to the signal strength of the reporter H.

The encoded regions may not only lie on the surface or the surface of the pores of a porous medium but may be an internal or integral part of the porous medium. For example, if the encoded regions were added intact (e.g., a particle of encoded material) during the synthesis of the porous medium, then the encoded region would be entrained within the porous medium and reside within the porous medium instead of on its surface. FIGS. 4A-4G illustrate an embodiment where the encoded regions 12a-12g reside or are embedded in the interior of the porous medium 10 where the elements shown therein are identified with the same reference characters used in the embodiment of FIGS. 3A-3F to identify similar and/or like elements shown therein. Similar to the encoded regions embodied in FIGS. FIGS. 3A-3F (and in FIGS. 1A-1D and 2A-2B), are in contact with the fluid moving through the porous medium 10. The encoded regions 12a-12g contact the fluid by convection, turbulent mixing or diffusion and the fluid within or on the encoded regions 12a-12g is mixed with the analyte-containing, bulk fluid. The reporter staining and determination of the analyte in this case is the same as when the encoded regions are on the surface of the porous medium 10 as illustrated in FIGS. 3A-3F.

One preferred method to encode the encoded region is to employ optical encoding where an emission of electromagnetic radiation that comprises a unique, exclusive and distinct optical code, optical barcode or spectral signature is induced by suitable excitation of the emitting species or emitters. For example, a simple type of optical encoding is illustrated in FIGS. 1A-1D, which show that the two encoded regions 12a and 12b within the porous medium 10 are optically encoded by their unique striping pattern (horizontal or vertical stripes). Therefore, one can distinguish the two different and separate encoded regions 12a and 12b by their stripe pattern. Once the encoded region 12a, 12b has been identified, the analyte 14a, 14b that binds to that particular region 12a, 12b is also known, since the identity of the bound analyte 14a, 14b was associated with encoded region 12a, 12b prior to the beginning of the analysis. Unless the analyte 18a, 18b was stained prior to the analysis (FIGS. 2A-2B), staining of the analyte 14a, 14b with a reporter stain 16, and measuring the strength of the reporter signal obtained for that particular encoded region 12a, 12b, provides information on the amount of that analyte 14a, 14b present in the fluid within the porous medium 10.

Optically encoding is advantageous in that the encoded regions need not be physically contacted to detect the electromagnetic radiation absorption, emission or reflection. Optical encoding may use electromagnetic radiation of any wavelength or type including x-rays, ultraviolet (UV), visible light, infrared (IR), microwave, radio waves or any other wavelength. The encoded regions may be identified optically by any type of absorption, emission, reflection or scattering of electromagnetic radiation or by the absence of said optical phenomena or combinations of these phenomena. Unique optical codes may be created for the encoded regions by using one or more emitting or absorbing species, or combinations of these species, to create unique multi-emitter or multi-absorber or multiple-scatterer optical codes for each encoded region.

A unique optical code for an encoded region may be created in many ways. One type of optical code that may be used to identify an encoded region is based on the presence or absence of one or more emitting or absorbing species to give a fingerprint or spectral signature which is binary in nature with respect to the number of different wavelengths comprising the optical code (i.e., a given color or wavelength is either present or absent). As an example of an encoded region with two color binary encoding using red and green—there are three codes possible: (i) red present and green present, (ii) red present and green absent and (iii) green present and red absent.

Instead of choice of either the presence or absence of a given wavelength, optical codes for encoded regions may be generated by varying the intensity of one or more emitters relative to other emitters in the same optical encoding material. Optical codes comprising one or more emitting or absorbing species can also provide encoded region differentiation by using optical codes based on the absolute intensity of the one or more emitters or absorbers. For example, for an optical code comprised of three IR emitters (materials which emit IR radiation under suitable excitation), where each emitter emits an absolute amount of light (i.e., photons per second per emitting volume measured at a detector), may be differentiated from other encoded regions which have different intensity for the three different emitters. A unique optical code or spectral signature may be created by determining the absolute relative intensity of these three emitters. When using absolute intensity encoding, a region with an absolute intensity (in arbitrary units) of the three emitters of 1:2:4 may be differentiated from an encoded region with an optical code of 2:4:8 for the same three emitters. The 1:2:4 may be differentiated from 2:4:8 because the 2:4:8 optical code is twice a bright (i.e., twice as high of a signal on the detector) because it is emitting twice as many photons per time per unit volume. In order to differentiate the 1:2:4 optical code from the 2:4:8 optical code, the absolute intensity of each emitter in the optical code must be determined. An absolute intensity measurement necessitates holding a wide variety of variables and data acquisition parameters, such as the power stability of excitation source and detector, the surface roughness of the sample, the angle of illumination, the sample to excitation and sample to detector distances and many other optical parameters, constant in order to obtain the exact number of photons emitted per time per emitting volume. When using the absolute intensity method of optically encoding the encoded regions, the number of absolute intensity optical codes (N_{A}) for a given resolvable intensity interval (I) and a given number of wavelengths (W) is N_{A}=I^{W}−1.

It also possible to generate distinguishable encoded regions by using optical codes based on ratiometric intensity encoding instead of or in addition to absolute intensity encoding. In ratiometric encoding, instead of measuring the absolute intensity of the different wavelengths, the optical code is created from the ratio of intensities among the various emission wavelengths. When generating absolute intensity optical codes discussed above, the optical codes are generated from the emission intensities of emitters of a, b and c—but in ratiometric encoding, the optical code is generated from the ratio of the intensities. For example, for the three emitter intensities a, b and c, there are two unique intensity ratios which are a:c and b:c. There are fewer ratiometric intensity optical codes than absolute intensity optical codes for the same interval resolution and number of wavelengths but, because of the substantial amelioration of the optical and instrumental issues discussed in the previous paragraph, the ratiometric intensity codes are far easier to resolve and accurately determine in practice. When using the ratiometric intensity method of optically encoding the encoded regions, the number of ratiometric optical codes (N_{R}) for given resolvable intensity ratio interval (I) (i.e., how many ratios may be resolved) and a given number of wavelengths (W) is N_{R}=I^{W-1}.

The materials used to generate the binary, absolute and ratiometric optical codes and the encoded regions may include Raman-active materials, organic dyes, nanoparticles, inorganic pigments, lanthanide and rare earth materials, quantum dots, vapor deposited thin film materials, photonic crystals or any combination in any ratio of these materials. The encoded regions could also be encoded with self-luminescent materials such as laser diodes, chemiluminescent materials, light emitting diodes, lasers, filaments or materials that transpond like RFID tags.

It is also possible to generate optical codes for the encoded regions by using any combination of two or three optical code-generating methods discussed above: (i) binary optical codes, (ii) absolute emission intensity optical codes and (iii) ratiometric intensity optical codes.

The following examples are not meant to limit the scope of the present disclosure.

### Examples

### Example 1

Three different shapes—a square, a circle and a triangle—are cut from sheets of an organic polymer (e.g., polyacrylic acid). These polymer shapes will form the encoded regions (in this case encoded by shape) within the porous medium. Capture probe 1 is attached to the square pieces of polymer, capture probe 2 is attached to the circular polymer pieces and capture probe 3 is attached to the triangular polymer pieces. Capture probe 1 specifically binds to analyte 1, capture probe 2 specifically binds to analyte 2 and capture probe 3 specifically bind to analyte 3. The polymer shapes with their specific capture probes are pooled together and cast into a melted agarose gel. When the gel hardens, the capture probe-derivatized polymer shapes constitute the encoded regions within the porous medium. A fluid, which contains dye-labeled analytes 1, 2 and 3, is passed through the porous agarose medium, contacts the encoded regions with their capture probes and the analytes within the fluid bind to their specific capture probes which are attached to their specific encoded region. The amount of dye label measured on each encoded region is proportional to the amount of analyte captured on that encoded region and proportional to the amount of analyte in the fluid.

### Example 2

Three different colored spheres—red, green and blue spheres—are formed from porous glass. These colored glass spheres will form the encoded regions (in this case encoded by color) within the porous medium. Capture probe 1 is attached to the red spheres, capture probe 2 is attached to the green spheres and capture probe 3 is attached to the blue spheres. Capture probe 1 specifically binds to analyte 1, capture probe 2 specifically binds to analyte 2 and capture probe 3 specifically bind to analyte 3. The colored spheres with their specific capture probes are pooled together and mixed with uncolored, transparent glass beads and placed into a column. The colored spheres form the encoded regions and the inter-sphere voids generated from the packing of all the spheres forms the pores of the porous medium. A fluid, which contains dye-labeled analytes 1, 2 and 3, is passed through the porous agarose medium, contacts the encoded regions with their capture probes and the analytes within the fluid bind to their specific capture probes which are attached to their specific encoded region. The amount of dye label measured on each encoded region is proportional to the amount of analyte captured on that encoded region and proportional to the amount of analyte in the fluid.

### Example 3

Three different size spheres—large, medium and small spheres—are formed from porous paper. These paper spheres will form the encoded regions (in this case encoded by size) within the porous medium. Capture probe 1 is attached to the large spheres, capture probe 2 is attached to the medium spheres and capture probe 3 is attached to the small spheres. Capture probe 1 specifically binds to analyte 1, capture probe 2 specifically binds to analyte 2 and capture probe 3 specifically bind to analyte 3. The different size spheres with their specific capture probes are pooled together, mixed with paper spheres of a size much smaller than the small, medium and large spheres (i.e., very small spheres) and placed into a column. The small, medium and large spheres form the encoded regions and the inter-sphere voids generated from the packing of all the spheres forms the pores of the porous medium. A first fluid, which contains unlabeled analytes 1, 2 and 3, is passed through the porous medium, contacts the encoded regions with their capture probes and the analytes within the fluid bind to their specific capture probes which are attached to their specific encoded region. A second fluid, which contains a reporter material that will stain all analytes bound to their respective encoded regions, is passed through the porous medium containing the encoded regions. A third fluid is passed through the porous medium to wash and remove any residual reporter material that was not bound specifically to the analyte. The amount of reporter label measured on each encoded region (i.e., the amount of reporter material on the small, medium and large spheres) is proportional to the amount of analyte captured on that encoded region and proportional to the amount of analyte in the first fluid.

### Example 4

Eight particle sets, P₁-P₈, are optically encoded using three different organic dyes in a binary fashion. The dyes, D₁, D₂ and D₃, are either present or absent within each of the eight particle sets P₁-P₈ according to the table below where 1=present and 0=absent.

| P | D₁ | D₂ | D₃ |
|---|---|---|---|
| P₁ | 0 | 0 | 0 |
| P₂ | 1 | 0 | 0 |
| P₃ | 0 | 1 | 0 |
| P₄ | 0 | 0 | 1 |
| P₅ | 1 | 1 | 0 |
| P₆ | 0 | 1 | 1 |
| P₇ | 1 | 0 | 1 |
| P₈ | 1 | 1 | 1 |

Next, capture probes C₁ through C₈ are attached to encoded particles P₁ through P₈, respectively. Capture probes C₁ through C₈ can selectively capture analytes A₁ through A₈, respectively, and exclude other analytes from capture. The analytes A₁ through A₈ are all labeled with a reporter material. The particle sets are derivatized with capture probes C₁ through C₈ are pooled and mixed with a nanocrystalline cellulose/solvent slurry and cast into a cylindrical tube. The particles P₁ through P₈ are randomly dispersed throughout the cellulose (porous medium) in the tube and become the encoded regions. A fluid, which contains some amounts of reporter-labeled analytes A₁ through A₈, is placed into one end of the tube and a pressure gradient imposed on the tube to cause the fluid to flow into, through and then out of the porous medium with encoded regions within the tube. As the fluid flows though the porous medium, the labeled analytes A₁ through A₈ (if present) selectively bind to their respective capture probes C₁ through C₈ on particle sets P₁ through P₈, respectively. Imaging the tube and contents determines the amount of reporter associated with each encoded region P₁ through P₈. The intensity of the reporter signal on each of the particle sets P₁ through P₈ is proportional to the amount of analytes A₁ through A₈ present in the fluid.

### Example 5

Eight particles sets, P₁-P₈, are optically encoded using two different color (i.e., two different emission or absorption spectra) inorganic pigments using an absolute intensity optical encoding method. The pigments, D₁ and D₂, are either present or absent and, when present, can take on intensity values of 1 or 2, and have an intensity value of 0 when absent. Since an absolute intensity optical encoding method is used, an intensity of 1 may be distinguished from an intensity of 2. When using the absolute intensity method of optically encoding the encoded regions, the number of absolute intensity optical codes (N_{A}) for a given resolvable intensity interval (I) and a given number of wavelengths (W) is N_{A}=I^{W}−1 or eight optical codes in this case. The “−1” in the equation for the number of absolute intensity optical codes corresponds to the “no code” material represented by the gray font line in the table below.

Since the 0,0 code has no absolute intensity, it is not defined and therefore the “−1” removes the 0,0 code from the number of possible codes to be used. Next, capture probes C₁ through C₈ are attached to encoded particle sets P₁ through P₈, respectively. Capture probes C₁ through C₈ can selectively capture analytes A₁ through A₈, respectively, and exclude other analytes from capture. The analytes A₁ through A₈ are all labeled with a reporter material. The eight derivatized particle sets with eight different capture probes C₁ through C₈ are pooled and mixed with a clear, porous polyurethane gel (porous medium) and cast into a film. The particle sets P₁ through P₈ (incipient encoded regions) are randomly dispersed throughout the cellulose (porous medium) in the tube and become the encoded regions. A fluid, which contains some amount of reporter-labeled analytes A₁ through A₈, is placed into one end of the tube and a partial vacuum (reduced pressure) applied to the opposite end of the tube to cause the fluid to flow into, through and then out of the porous medium with encoded regions within the tube. As the fluid flows though the porous medium, the labeled analytes A₁ through A₈ (if present) selectively bind to their respective capture probes C₁ through C₈ on particle sets P₁ through P₈, respectively. Imaging the film and contents determines the amount of reporter associated with each encoded region P₁ through P₈. The intensity of the reporter signal on each of the particle sets P₁ through P₈ is proportional to the amount of analytes A₁ through A₈ present in the fluid.

### Example 6

Nine particle sets, P₁-P₉, are optically encoded using three different rare earth (i.e., lanthanide) emitters (which provide three different emission or absorption spectra, that are distinguishable from one another, corresponding to the three lanthanide emitters) using a ratiometric intensity optical encoding method. The three lanthanide emitters, D₁, D₂ and D₃, provide two distinct and unique ratios D₁/D₃ and D₂/D₃. Each encoded region has three rare earth emitters associated with it but is identified only by its ratiometric optical code. In this example, there are three resolvable D₁/D₃ intensity ratios and three resolvable D₂/D₃ ratios as shown in the table below.

| P | D₁/D₃ | D₂/D₃ |
|---|---|---|
| P₁ | 1 | 1 |
| P₂ | 2 | 2 |
| P₃ | 3 | 3 |

When using the ratiometric intensity method of optically encoding the encoded regions, the number of ratiometric optical codes (N_{R}) for given resolvable intensity ratio interval (I) (i.e., how many ratios may be resolved between two encoding variables which are the lanthanide emitters in this example) and a given number of wavelengths (W) is N_{R}=I^{W-1}. In this example, for three resolvable intensity ratios (I=3) and three wavelengths (W=3), there are N_{R}=I^{W-1}=3³⁻¹=9 optical codes available to encode the encoded regions. Thus, the nine optical codes corresponding to nine encoded regions are the nine D₁/D₃ and D₂/D₃ designators, namely (see table above) P₁=1,1; P₂=1,2; P₃=1,3; P₄=2,1; P₅=2,2; P₆=2,3; P₇=3,1; P₈=3,2; and P₉=3,3. Next, capture probes C₁ through C₉ are attached to encoded particle sets P₁ through P₈, respectively. Capture probes C₁ through C₉ can selectively capture analytes A₁ through A₉, respectively, and exclude other analytes from capture. The analytes A₁ through A₉ are all labeled with a reporter material. The nine derivatized particle sets with nine different capture probes C₁ through C₉ are pooled together and spread onto a lamellar nitrocellulose membrane (porous medium). The particle sets P₁ through P₉ (incipient encoded regions) are randomly dispersed over the nitrocellulose membrane (porous medium) and become the encoded regions. A fluid, which contains none, one or more of the reporter-labeled analytes A₁ through A₉, is placed onto one end or side of the membrane (proximal) and an absorbent material placed at the other (distal). This causes the fluid to move through the porous medium from the proximal end toward the distal end and then out of the porous medium which contain the encoded regions. As the fluid flows though the porous medium and contacts the encoded regions, the labeled analytes A₁ through A₉ (if present) selectively bind to their respective capture probes C₁ through C₉ on particle sets P₁ through P₉, respectively. Imaging the film and contents determines the amount of reporter associated with each encoded region P₁ through P₉. The intensity of the reporter signal on each of the particle sets P₁ through P₈ is proportional to the amount of analytes A₁ through A₉ present in the fluid.

It should be understood by those skilled in the art that in addition to the embodiments and examples disclosed herein, there are a very large number of other types of porous media, encoded regions, methods of preparing encoded regions within porous media, methods of encoding the encoded regions, methods to determine the amount of analyte present, methods of moving fluids, methods of determining the precise nature of the encoded region and its associated reporter level as well as devices, reporter materials, protocols and other combinations of reporters that fall within the scope of the present disclosure and claims.

1. A method for sensing, identifying, and characterizing one or more analytes in a fluid, the method comprising:
preparing a porous medium comprising one or more pores, the porous medium including one or more encoded regions, wherein the one or more encoded regions:
contact the internal pore surface or surfaces, external surface or surfaces, or any combination thereof, of the porous medium;
comprises a capture probe or binding element which selectively captures the one or more of the analytes in the fluid; and
comprises at least one of physical properties and chemical properties which are different from the other encoded regions;
filling the pores of the porous medium with the fluid containing the one or more analytes, the one or more analytes contacting the encoded regions of the porous medium, wherein the capture probe or binding element of each encoded region selectively captures the one or more analytes; and
characterizing the one or more analytes captured by each encoded region.

2. The method of claim 1, wherein the porous medium is made from a material comprising an organic polymer, a glass, a mineral, a paper, a wood, an oxide, a carbohydrate, agarose, a metal or any combination thereof.

3. The method of claim 1, wherein encoding of the encoded regions is based on a physical property, a chemical property, a magnetic property, or any combination thereof.

4. The method of claim 3, wherein the physical property comprises a shape, a size, a color, emission or absorption behavior, the number of voids, the number of indentations, the number of channels, the number of markings or protrusions, the scattering of electromagnetic radiation, or any combination thereof.

5. The method of claim 1, wherein each of the analytes comprises an organic molecule, a biochemical molecule, a nucleic acid, a peptide, a protein, an antibody, a drug candidate molecule, an anionic and cationic inorganic material, a radioactive species, or any combination thereof.

6. The method of claim 1, wherein an encoding material is used for generating binary, absolute and ratiometric optical codes within the encoded regions, or any combination thereof.

7. The method of claim 6, wherein the encoding material comprises a Raman-active material, an organic dye, an inorganic pigment, a lanthanide material, a rare earth material, quantum dots, a vapor deposited thin film material, photonic crystals, or any combination of thereof in any ratio.

8. The method of claim 1, wherein the encoded regions are encoded with self-luminescent materials including laser diodes, chemiluminescent light emitting diodes, lasers, filaments or materials that transpond like RFID tags, or any combination thereof.

9. The method of claim 1, wherein the characterizing of the one or more analytes includes determining the amount of the one or more analytes captured by each encoded region, wherein the determining the amount of the one or more analytes comprises:
contacting a reporter material with the one or more analytes captured with the capture probe or binding element of each encoded region; and
measuring the level of the reporter material on each encoded region to determine the amount of each of the one or more analytes bound to the capture probe or binding element of that encoded region.

10. The method of claim 9, wherein the contacting of the reporter material with the one or more analytes comprises labeling, attaching or derivitizing the one or more analytes with the reporter material.

11. A system for sensing, identifying, and characterizing one or more analytes in a fluid, the system comprising:
a porous medium comprising one or more pores;
one or more encoded regions contacting internal pore surface or surfaces, external surface or surfaces, and any combination thereof, of the porous medium, and wherein the one or more encoded regions comprises at least one of physical properties and chemical properties which are different from the other encoded regions; and
a capture probe or binding element disposed on or in the one or more encoded regions, the capture probe or binding element for selectively capturing the one or more of the analytes in the fluid;
wherein in operation, the pores of the porous medium are filled with the fluid containing the one or more analytes, the one or more analytes contacting the encoded regions of the porous medium;
wherein the capture probe or binding element of each encoded region selectively captures the one or more analytes; and
wherein the character of the one or more analytes captured by each encoded region is determined.

12. The system of claim 11, wherein the porous medium is made from a material comprising an organic polymer, a glass, a mineral, a paper, a wood, an oxide, a carbohydrate, agarose, a metal or any combination thereof.

13. The system of claim 11, wherein encoding of the encoded regions is based on a physical property, a chemical property, a magnetic property, or any combination thereof.

14. The system of claim 13, wherein the physical property comprises a shape, a size, a color, emission or absorption behavior, the number of voids, the number of indentations, the number of channels, the number of markings or protrusions, the scattering of electromagnetic radiation, or a any combination thereof.

15. The system of claim 11, wherein each of the analytes comprises an organic molecule, a biochemical molecule, a nucleic acid, a peptide, a protein, an antibody, a drug candidate molecule, an anionic and cationic inorganic material, a radioactive species, or any combination thereof.

16. The system of claim 11, wherein an encoding material is used for generating binary, absolute and ratiometric optical codes within the encoded regions, or any combination thereof.

17. The system of claim 16, wherein the encoding material comprises a Raman-active material, an organic dye, an inorganic pigment, a lanthanide material, a rare earth material, quantum dots, a vapor deposited thin film material, photonic crystals, or any combination of thereof in any ratio.

18. The system of claim 11, wherein the encoded regions are encoded with self-luminescent materials including laser diodes, chemiluminescent light emitting diodes, lasers, filaments or materials that transpond like RFID tags, or any combination thereof.

19. The system of claim 11, wherein the characterizing of the one or more analytes includes determining the amount of the one or more analytes captured by each encoded region, the determining the amount of the one or more analytes comprises:
contacting a reporter material with the one or more analytes captured with the capture probe or binding element of each encoded region; and
measuring the level of the reporter material on each encoded region to determine the amount of each of the one or more analytes bound to the capture probe or binding element of that encoded region.

20. The system of claim 19, wherein the contacting of the reporter material with the one or more analytes comprises labeling, attaching or derivitizing the one or more analytes with the reporter material.

21. The method of claim 1, wherein the one or more encoded regions are encoded with one or more lanthanide materials.

22. The system of claim 11, wherein the one or more encoded regions are encoded with one or more lanthanide materials.

23. The system of claim 11, further comprising a reporter material for determining the amount of each of the one or more analytes bound to the capture probe or binding element of that encoded region.
